(19) United States
(12) Patent Application Publication
(10) Pub. No.: US 2022/0125690 A1
(43) Pub. Date: Apr. 28, 2022

(54) EXTERNAL AGENT FOR SKIN OR MUCOUS MEMBRANE AND PRODUCTION METHOD THEREOF, AND BASE FOR EXTERNAL AGENT FOR SKIN OR MUCOUS MEMBRANE

(71) Applicants: Naruhito MATSUDA, Tokyo (JP); KANAGAWA UNIVERSITY, Yokohama-shi, Kanagawa (JP)

(72) Inventors: Kazuo TAJIMA, Yokohama-shi, Kanagawa (JP); Yoko IMAI, Yokohama-shi, Kanagawa (JP); Kana MIYASAKA, Yokohama-shi, Kanagawa (JP); Naruhito MATSUDA, Tokyo (JP)

(21) Appl. No.: 17/427,341

(22) PCT Filed: Jan. 30, 2020
(86) PCT No.: PCT/JP2020/003497

§ 371 Date: Jul. 30, 2021

(30) Foreign Application Priority Data
Jan. 30, 2019 (JP) 2019-014661

(51) Int. Cl. A61K 8/06; A61Q 19/00; A61K 8/31; A61K 47/06; A61K 9/00; A61K 9/06; A61K 9/107

(52) U.S. Cl. CPC A61K 8/062; A61Q 19/00; A61K 8/31; A61K 9/107; A61K 9/0014; A61K 9/06; A61K 47/06

(57) ABSTRACT

Provided is an external agent for skin or mucous membrane excellent in feeling during use despite containing petrolatum as a base. An external agent for skin or mucous membrane according to the present invention includes an oil phase, as an inner phase, that consists of petrolatum or is a liquid or a semi-solid including petrolatum with a viscosity of 5000 mPa·s or more at 25° C. and a water-insoluble functional component phase, as an inner phase, including a water-insoluble functional component, and an aqueous phase as an outer phase, in which particles of a polycondensation polymer having a hydroxyl group and/or vesicles formed of an amphiphilic substance are present at an interface between the oil phase and the aqueous phase, and at an interface between the functional component phase and the aqueous phase.

### TECHNICAL FIELD

The present invention relates to an external agent for skin or mucous membrane and a method for producing an external agent for skin or mucous membrane, and a base for an external agent for skin or mucous membrane.

### BACKGROUND ART

Petrolatum is in widespread use as a base for external agents for skin in the field of cosmetics and pharmaceuticals, etc., because of its excellent safety as well as excellent skin protecting effects and moisturizing effects. Currently, products prepared by addition and blending of a functional component to the petrolatum are provided as such an external agent for skin.

However, most of functional components added to the petrolatum are insoluble in petrolatum, and thus exist under solid condition in the external agent for skin. The presence of such a solid component in the external agent for skin can cause a feeling of surface-roughness when the external agent for skin is applied to the skin.

On the other hand, when the petrolatum itself is applied to the skin, the petrolatum has poor spreadability on the skin surface, and causes strong stickiness.

For improving the feeling during use of such external agents for skin containing the petrolatum as the base, for example, Patent Document 1 reports an external agent that contains a high concentration of petrolatum and has a specifically adjusted melting point and stringiness value. This specific adjustment of the melting point and the stringiness value can suppress the stickiness of the external agent, which is caused by the petrolatum. However, in such an external agent, the petrolatum is merely diluted with a liquid oil agent. Such an external agent somewhat improves the spreadability on the skin surface, but the external agent is unable to suppress the feeling of stickiness due to direct contact of the oily substance including the petrolatum with the skin upon the application of the external agent. Additionally, the external agent provides no suppression of the surface-roughness, and the external agent is not necessarily sufficient in terms of the feeling during use. Moreover, in some cases, the sensitivity of the skin against the oil agent added to a liquefied petrolatum is a matter of concern.

Patent Document 1: Japanese Unexamined Patent Application, Publication No. 2016-222585

### DISCLOSURE OF THE INVENTION

### Problems to be Solved by the Invention

The present invention was developed in view of the abovementioned situation, and an object of the present invention is to provide an external agent for skin or mucous membrane excellent in feeling during use despite containing petrolatum as a base.

### Means for Solving the Problems

The present inventors made a series of intensive studies for solving the problems described above. As a result, the inventors have found to provide an external agent for skin or mucous membrane excellent in feeling during use despite containing petrolatum as a base by emulsifying petrolatum and a functional component using specific particles of a polycondensation polymer or vesicles, to accomplish the present invention. Specifically, the present invention provides the following.

A first aspect of the present invention relates to an external agent for skin or mucous membrane, including: an oil phase, as an inner phase, that consists of petrolatum or is a liquid or a semi-solid including petrolatum with a viscosity of 5000 mPa·s or more at 25° C., and a water-insoluble functional component phase, as an inner phase, including a water-insoluble functional component, and an aqueous phase as an outer phase, in which particles of a polycondensation polymer having a hydroxyl group and/or vesicles formed of an amphiphilic substance are present at an interface between the oil phase and the aqueous phase, and at an interface between the water-insoluble functional component phase and the aqueous phase.

A second aspect of the present invention relates to an external agent for skin or mucous membrane, including: an oil phase, as an inner phase, that consists of petrolatum and a water-insoluble functional component or is a liquid or a semi-solid including petrolatum and a water-insoluble functional component with a viscosity of 5000 mPa·s or more at 25° C., and an aqueous phase as an outer phase, in which particles of a polycondensation polymer having a hydroxyl group and/or vesicles formed of an amphiphilic substance are present at an interface between the oil phase and the aqueous phase.

A third aspect of the present invention relates to the external agent for skin or mucous membrane according to the first or second aspect of the present invention, in which a content of the water-insoluble functional component with respect to a total amount of the external agent for skin or mucous membrane is 50% by mass or less.

A fourth aspect of the present invention relates to the external agent for skin or mucous membrane according to any one of the first to third aspects of the present invention, in which the water-insoluble functional component is a functional component of cosmetics and/or a functional component of pharmaceuticals.

A fifth aspect of the present invention relates to an external agent for skin or mucous membrane, including: an oil phase, as an inner phase, that consists of petrolatum or is a liquid or a semi-solid including petrolatum with a viscosity of 5000 mPa·s or more at 25° C., and an aqueous phase, as an outer phase, including a water-soluble functional component, in which particles of a polycondensation polymer having a hydroxyl group and/or vesicles formed of an amphiphilic substance are present at an interface between the oil phase and the aqueous phase.

A sixth aspect of the present invention relates to the external agent for skin or mucous membrane according to the fifth aspect of the present invention, in which a content of the water-soluble functional component with respect to a total amount of the external agent for skin or mucous membrane is 0.001% by mass or more and 50% by mass or less.

A seventh aspect of the present invention relates to the external agent for skin or mucous membrane according to the fifth or sixth aspect of the present invention, in which the water-insoluble functional component is a functional component of cosmetics and/or a functional component of pharmaceuticals.

An eighth aspect of the present invention relates to the external agent for skin or mucous membrane according to any one of the first to seventh aspects of the present invention, in which a content of the oil phase with respect to a total amount of the external agent for skin or mucous membrane is 70% by mass or less.

A ninth aspect of the present invention relates to the external agent for skin or mucous membrane according to any one of the first to eighth aspects of the present invention, in which the content of the petrolatum with respect to a total amount of the external agent for skin or mucous membrane is 2% by mass or more and 70% by mass or less.

A tenth aspect of the present invention relates to a base for an external agent for skin or mucous membrane to produce the external agent for skin or mucous membrane by addition of a water-soluble functional component and/or a water-insoluble functional component, the base including: an oil phase, as an inner phase, that consists of petrolatum or is a liquid or a semi-solid including petrolatum with a viscosity of 5000 mPa·s or more at 25° C., and an aqueous phase as an outer phase, and particles of a polycondensation polymer having a hydroxyl group and/or vesicles formed of an amphiphilic substance, in which a part of the particles and/or the vesicles are present at an interface between the oil phase and the aqueous phase.

An eleventh aspect of the present invention relates to the base for an external agent for skin or mucous membrane according to the tenth aspect of the present invention, in which the external agent for skin or mucous membrane is an ointment.

A twelfth aspect of the present invention relates to a method for producing an external agent for skin or mucous membrane, the method including: adding a water-soluble functional component and/or a water-insoluble functional component to a base for an external agent for skin or mucous membrane, and mixing the water-soluble functional component and/or the water-insoluble functional component with the base, in which the base includes: an oil phase, as an inner phase, that consists of petrolatum or is a liquid or a semi-solid including petrolatum with a viscosity of 5000 mPa·s or more at 25° C., and an aqueous phase as an outer phase, and particles of a polycondensation polymer having a hydroxyl group and/or vesicles formed of an amphiphilic substance, in which a part of the particles and/or the vesicles are present at an interface between the oil phase and the aqueous phase, and at an interface between the functional component phase and the aqueous phase.

### Effects of the Invention

According to the present invention, an external agent for skin or mucous membrane excellent in feeling during use despite containing petrolatum as a base can be produced.

### BRIEF DESCRIPTION OF THE DRAWINGS

FIG. 1 shows a microscopy image of base 1.

FIG. 2 shows a microscopy image of base 2.

FIG. 3 shows a microscopy image of the sample obtained in Example 1.

FIG. 4 shows a microscopy image of allantoin powder.

FIG. 5 shows a microscopy image of the sample obtained in Example 2.

FIG. 6 shows a microscopy image of cerebroside powder.

FIG. 7 shows a microscopy image of the sample obtained in Example 3.

FIG. 8 shows a microscopy image of an aqueous dispersion of 32% by mass titanium oxide.

FIG. 9 shows a microscopy image of the sample obtained in Example 4.

FIG. 10 shows a microscopy image of a silicone oil dispersion of 29% by mass titanium oxide.

### PREFERRED MODE FOR CARRYING OUT THE INVENTION

Hereinafter, embodiments of the present invention will be explained, but the present invention is not limited thereto.

All of the external agents for skin or mucous membrane described hereinafter are an oil-in-water emulsion (O/W type emulsion) in which an oil phase is dispersed in an aqueous phase. For convenience, the term “emulsification” is used herein to include not only the concept of dispersing a liquid (inner phase) into water (outer phase), but also the concept of dispersing a solid into water. Moreover, in the latter case, the solid shall be referred to as the “inner phase” and the water as the “outer phase”. Furthermore, the “inner phase” and the “outer phase” as used herein do not necessarily have to be a single physicochemical phase respectively, and may be, for example, a mixture in which a plurality of phases are present in one “inner phase” (one emulsion particle).

Further, the term “water-soluble” as used herein refers to having a solubility of 1 g/kg or more in water at 25° C. in an environment of 25° C. under atmospheric pressure. On the other hand, the term “water-insoluble” as used herein refers to having a solubility of less than 1 g/kg in water at 25° C.

Furthermore, the term “functional component” as used herein refers to a component that is added for the purpose of achieving various types of functions of the external agent for skin or mucous membrane. For example, the “functional component” in cosmetics includes whitening components, pH adjusters, ultraviolet protective agents, abrasives, antibacterial agents, fragrances, colorants, antioxidants, moisturizers, thickeners, preservatives, and the like, and the “functional component” in pharmaceuticals includes active ingredients, moisturizers, antioxidants, pH adjusters, thickeners, preservative, and the like.

More specifically, additives for cosmetics are not particularly limited, and examples of the additives for cosmetics include 1,2-octane diol (caprylyl glycol), 1,2-hexanediol, 1,2-pentanediol (pentylene glycol), 1,3-butylene glycol (BG), α-hydroxy acid (glycolic acid), sodium dl-α-tocopheryl phosphate, sodium DL-pyrrolidone carboxylate solutions (PCN-Na), dipropylene glycol, human oligopeptides (EGF), L-aspartic acid, L-alanine, L-arginine, L-isoleucine, L-oxyproline (hydroxyproline), L-glutamic acid, L-threonine, L-serine, L-tyrosine, L-valine, L-histidine, L-histidine hydrochloride, L-phenylalanine, L-proline, L-lysine solutions, L-leucine, N-acetyl-L-hydroxyproline, N-(hexadecyloxyhydroxypropyl)-N-hydroxyethylhexadecanamide (cetyl PG hydroxyethyl palmitamide), PEGs (PEG 4, 6, 8, 12, etc.), propylene glycol (1,2-propanediol), Galactomyces ferment filtrate, Rehmannia Chinensis root extracts (Rehmannia root extracts, Rehmannia extract), acrylamide-acrylic acid-dimethyldiallylammonium chloride copolymer solutions (polyquaternium 39), Angelica keiskei (Ashitaba) extracts, Angelica keiskei (Ashitaba) leaf/stem extracts, diglyceryl adipate mixed fatty acid ester (bis-diglyceryl polyacyladipate-2), asparagus stem extracts, sodium acetylated hyaluronate, Hydrangea serrata (hydrangea) leaf extracts, aminobutyric acid, Alcaligenes polysaccharides, Algae extracts (MARINE PURGE, seaweed extracts 1, seaweed extracts 4), Althea extracts, Althea root extracts, Aloe extracts (2), Aloe vera juice, Aloe vera leaf extracts, Prunus armeniaca (apricot) kernel extracts, Rosa roxburghii fruit extracts, Asarum sieboldi rhizome/root extracts, Citrus unshiu peel extracts, Rose fruit extracts, ectoine, ethylhexylglycerin, dimethyldiallylammonium chloride-acrylic acid copolymer solution (polyquaternium-22), levocarnitine chloride, lysine hydrochloride, Phellodendron amurense extracts, Coptis japonica extracts, Coptis japonica root extracts, Abelmoschus esculentus (okra) extracts, Abelmoschus esculentus (okra) fruit extracts, Nasturtium officinale extracts, Nasturtium officinale leaf extracts, Nasturtium officinale leaf/stem extracts, orange extracts, orange fruit extracts, hot spring water, seawater, hydrolyzed elastin, hydrolyzed keratin (wool), hydrolyzed keratin solutions, hydrolyzed collagen, hydrolyzed collagen solutions, hydrolyzed collagen powder, hydrolyzed conchiolin, hydrolyzed conchiolin solutions, hydrolyzed silk, hydrolyzed silk solutions, hydrolyzed silk powder, hydrogenated starch hydrolysate, hydrolyzed hyaluronic acid, hydrolyzed egg shell membrane, hydrolyzed albumen, Pueraria lobata root extracts, Rosa canina fruit extracts, Rubus idaeus (raspberry) extracts, kiwi extracts, Xylito (xylitol), Phellodendron amurense bark extracts, cucumber extracts, Cucumis sativus (cucumber) fruit extracts, apricot kernel extracts, Pueraria thunbergiana root extracts, Gardenia florida (gardenia) extracts, Gardenia florida (gardenia) fruit extracts, glycosyl trehalose, glycine, glycerin, glucose, glucosyl ceramide, sodium glutamate, grapefruit extracts, grapefruit fruit extracts, Chlorella vulgaris extracts, mulberry extracts, Gentiana lutea (gentian) root extracts, Gentiana lutea (gentian) rhizome/root extracts, Arctium lappa (burdock) extracts, Arctium lappa (burdock) root extracts, Oryza sativa (rice) bran extracts, Oryza sativa (rice) bran glycosphingolipid (rice ceramide), Oryza sativa (rice) bran fermentation extracts (Oryza sativa (rice) bran fermentation filtrate), cholesterol, Averrhoa carambola leaf extracts, sodium chondroitin sulfate, Asiasarum root extracts, umbilical extracts, succinoyl atelocollagen, Crataegus cuneata (hawthorn) fruit extracts, diglycerin, Perilla ocymoides leaf extracts (Perilla ocymoides extracts 1, iPerilla ocymoides extracts 2), dipropylene glycol (DPG), Paeonia albiflora (peony) extracts, Paeonia albiflora (peony) root extracts, sodium lysine dilauroyl glutamate (Pellicer), birch extracts (birch bark extract, Betula platyphylla japonica bark extracts), Tremella fuciformis polysaccharide, Lonicera japonica (honeysuckle) extracts, Lonicera japonica (honeysuckle) flower extracts, soluble collagen (soluble collagen 1, soluble collagen 3, soluble collagen 4), soluble proteoglycan (proteoglycan), Equisetum arvense extracts, sucrose, starfruit leaf extracts, glycosphingolipids, Pinus sylvestris cone extract, Hedera helix (ivy) extracts, Hedera helix (ivy) leaf/stem extracts, Malva sylvestris (mallow) extracts, Malva sylvestris (mallow) flower extracts, ceramide, sericin (hydrolyzed silk powder), sorbit solutions (sorbitol), Glycine soja (soybean) extracts, Glycine soja (soybean) seed extracts, soybean lysophospholipid solutions (lysolecithin), soybean phospholipid (lecithin), Jujube (Zizyphus jujuba) extracts (Ziziphus jujuba fruit extracts), Thyme extracts (1) (Thymus serpyllum extracts), Thyme extracts (2) (Thymus vulgaris flower/leaf extracts), taurine, Rosa damascena flower extracts, Polianthes tuberosa polysaccharide (Polianthes tuberosa polysaccharide solutions), Eugenia caryophyllus (clove) flower extracts, Citrus unshiu peel extracts, dextrin, Prunus persica kernel extracts, Zea mays (corn) kernel extracts, Solanum lycopersicum (tomato) extracts, Solanum lycopersicum (tomato) fruit extracts, trimethylglycine (betaine), trehalose (trehalose solutions), niacinamide (nicotinamide), lactic acid, sodium lactate, urea, Allium sativum (garlic) extracts, Allium sativum (garlic) bulb extracts, Rosa multiflora fruit extracts (Rose fruit extracts), Rosa canina extracts (Rosa canina fruit extracts, rosehip extracts), Hibiscus sabdariffa (hibiscus) flower extracts, Carum petroselinum (parsley) extracts, Coix lacryma-jobi ma-yuen seed extracts, honey, sodium hyaluronate, biotin, histidine, histidine HCl, bifidobacteria fermentation extracts (bifidobacteria extracts), Poria cocos sclerotium extracts, beech extracts, placental extracts, prune enzymolysis product (hydrolyzed Prunus domestica), propanediol, Paeonia suffruticosa root extracts, Humulus lupulus (hops) extracts, Humulus lupulus (hops) flower extracts, Humulus lupulus (hops) powder, polyquaterniums (Lipidure, polyquatemium-51, polyquaternium-61, 2-methacryloyloxyethyl phosphorylcholine-butyl methacrylate copolymer solutions, 2-methacryloyloxyethyl phosphorylcholine-stearyl methacrylate copolymer), Morus alba root extracts, Pinus sylvestris (pine) cone extracts, Lilium candidum bulb extracts, Origanum majorana (marjoram) extracts, Origanum majorana (marjoram) leaf extracts, maltitol, mannose, Sapindus mukorossi (soapberry) extracts, Sapindus mukorossi (soapberry) peel extracts, methyl gluceths (POE methylglucoside), Eucalyptus globulus (eucalyptus) extracts, Eucalyptus globulus (eucalyptus) leaf extracts, Saxifraga sarmentosa (saxifrage) extracts, Citrus junos (yazu) extracts, Citrus junos (yazu) fruit extracts, Citrus junos (yazu) seed extracts, Citrus junos (yazu) ceramide, ubiquinone (coenzyme Q10), Lilium candidum (lily) bulb extracts, Betula alba bark extracts, Fagus sylvatica bud extracts, coix seed extracts (Coix lacryma-jobi ma-yuen seed extracts), RICEPOWER No. 11 (rice extract No. 11), raffinose, Pyrus malus (apple) extracts, Pyrus malus (apple) fruit extracts, Ganoderma lucidum (mushroom) stem extracts, Ganoderma lucidum (mushroom) extracts, Lactuca scariola sativa (lettuce) extracts (1), Lactuca scariola sativa (lettuce) leaf extracts, Citrus limon (lemon) extracts, Citrus limon (lemon) fruit extracts, Astragalus sinicus extracts, royal jelly extracts (royal jelly), Persea gratissima (avocado) oils, Linum usitatissimum (linseed) seed oils, Prunus amygdalus dulcis (sweet almond) oils, Argania spinosa kernel oils (argan oils), Perilla frutescens oils, Silybum marianum seed oils, Olea europaea (olive) fruit oil, refined Olea europaea (olive) fruit oil, Theobroma cacao (cocoa) seed butter, apricot kernel oils, Aleurites moluccanus seed oils, Passiflora edulis seed oils (passion fruit), walnut oils, grape seed oils (Vitis vinifera seed oils), coconut oils (Cocos nucifera oils), Sesamum indicum (sesame) seed oils, Triticum vulgare (wheat) germ oils, Oryza sativa (rice) bran oils, Oryza sativa (rice) germ oils, Zea mays (corn) oils, safflower oils (Carthamus tinctorius seed oils), safflower oils, shea butter, Glycine soja (soybean) oils, Camellia sinensis (camellia) seed oils, Oenothera biennis (evening primrose) Oils, Camellia japonica seed oils, rapeseed oils, Coix lacryma-jobi ma-yuen (adlay) seed oils, Arachis hypogaea (peanut) oils, Ricinus communis (castor) oils, Helianthus annuus (sunflower) seed oils, Prunus domestica (prune) seed oils, Simmondsia chinensis (jojoba) oils, Macadamia ternifolia (macadamia nut) seed butter, Macadamia ternifolia (macadamia nut) seed oils, Gossypium herbaceum (cotton) seed oils, Citrus junos (yazu) seed oils, Camellia oleifera seed oils, Borago officinalis seed oils (borage seed oils), red palm oil, Rosa canina fruit oils (rosehip oils), horse fats, fish oils, Persea gratissima (avocado) oils, Copernicia cerifera (carnauba) wax, Euphorbia cerifera (candelilla) wax, Oryza sativa (rice) bran wax, beeswax, white wax (bleached beeswax), isododecane, isohexadecane, squalane, paraffins, polybutene, microcrystalline wax, petrolatum, light liquid isoparaffins, liquid isoparaffins, liquid paraffins, astaxanthin, arbutin, elastin, resveratrol, iron oxides, titanium oxides, zinc oxides, kaolin, mica, sericite, acrylates/C10-30 alkyl acrylate crosspolymers, ascorbic acid, acetyl hexapeptide-3, disodium adenosine triphosphate, allantoin, Arnica montana (arnica) flower extracts, hydrolyzed yeast extracts, Chamomilla recutita (matricaria) extracts, xanthan gum, dipotassium glycyrrhizate, stearyl glycyrrhetinate, salicylic acid, platinum (platinum nanocolloids), retinyl palmitate, panthenol, water-soluble vitamins, fat-soluble vitamins, fullerene, and the like.

The active ingredients for pharmaceuticals (excluding petrolatum as a skin moisturizer) are not particularly limited, and examples the active ingredients include: gentamicin sulfate and bacitracin-fradiomycin sulfate combination as agents, which prevention wound infection, in the external agent for skin; clobetasol propionate, diflorasone acetate, betamethasone dipropionate, difluprednate, fluocinonide, diflucortolone valerate, amcinonide, hydrocortisone butyrate propionate, betamethasone butyrate propionate, mometasone furoate, dexamethasone propionate, betamethasone valerate, beclomethasone dipropionate, dexamethasone valerate, fluocinolone acetonide, deprodone propionate, prednisolone valerate acetate, triamcinolone acetonide, hydrocortisone butyrate, clobetasone butyrate, alclometasone dipropionate, dexamethasone, hydrocortisone, and fludroxycortide as a corticosteroid preparation; ibuprofen piconol, suprofen, bendazac, ufenamate, and glycyrrhetinic acid as a nonsteroidal anti-inflammatory external agent; crotamiton, and crotamiton-hydrocortisone combination as an antipruritic; tacrolimus hydrate as a therapeutic agent for atopic dermatitis; methoxsalen as a therapeutic agent for vitiligo; nadifloxacin, adapalene, and benzoyl peroxide as a therapeutic agent for acne (therapeutic agent for pimples); tacalcitol hydrate, calcipotriol, maxacalcitol, calcipotriol hydrate-betamethasone dipropionate, vitamin A, and salicylic acid as a therapeutic agent for keratosis and/or psoriasis; azulene, lysozyme hydrochloride, bucladesine sodium, white soft sugar-povidone iodine, sulfadiazine, silver sulfadiazine, tretinoin tocoferil, mixed killed bacterial suspension-hydrocortisone, and zinc oxide ointment as a therapeutic agent for alopecia, as a therapeutic agent for skin ulcer; heparin sodium, and heparin as a blood circulation stimulant and skin moisturizer; and bimatoprost as a therapeutic agent for madarosis; and the like.

### «External Agent for Skin or Mucous Membrane According to First Aspect»

An external agent for skin or mucous membrane according to a first aspect is characterized by including an oil phase, as an inner phase, that consists of petrolatum or is a liquid or a semi-solid including petrolatum with a viscosity of 5000 mPa·s or more at 25° C., and a water-insoluble functional component phase, as an inner phase, including a water-insoluble functional component, and an aqueous phase as an outer phase, in which particles of a polycondensation polymer having a hydroxyl group and/or vesicles formed of an amphiphilic substance are present at the interface between the oil phase and the aqueous phase, and at the interface between the water-insoluble functional component phase and the aqueous phase.

In such an external agent for skin or mucous membrane, the oil phase and the water-insoluble functional component phase each constitute separate emulsion particles, and each constitute inner phases of different compositions. In other words, in such an external agent for skin or mucous membrane, at least two types of emulsion particles (inner phase), i.e. the oil phase and the water-insoluble functional component phase, are emulsified in the aqueous phase as the outer phase. More specifically, the water-insoluble functional component is not present in the oil phase, and a phase containing the petrolatum is not present in the water-insoluble functional component phase. The two phases are at least not in contact or mixed with each other, and they are separated from each other by at least the polycondensation polymer particles-vesicles phase/the aqueous phase/the polycondensation polymer particles-vesicles phase. However, this does not preclude the existence of an emulsion particle that contains both the petrolatum and the water-insoluble functional component.

### <Polycondensation Polymer Particles, and Vesicles>

The polycondensation polymer particles and the vesicles are present (i.e., interpose) at the interface between the oil phase and the aqueous phase, and at the interface between the functional component phase and the aqueous phase, and constitute an emulsified state via van der Waals force, and thus it can constitute a favorable emulsion regardless of the chemical composition, surface conditions, etc. of the aqueous phase, the oil phase and the functional substance.

It has been found that such an emulsification method enables the petrolatum, which is highly viscous at ambient temperature, to be emulsified in an extremely stable manner, and that the emulsion structure in which the polycondensation polymer particles or the vesicles physically coat the oil phase containing the petrolatum can reduce the viscosity of the external agent for skin or mucous membrane. When such an external agent for skin or mucous membrane is spread over the skin or mucous membrane by applying an external force through coating, etc., the aqueous phase is present between the oil phase or the water-insoluble functional component phase and the skin or mucous membrane, and thus direct contact of these phases with the skin or mucous membrane can be suppressed, leading to reduction of the feeling of stickiness on the skin or mucous membrane. In addition, even when the functional substance is in solid form, the functional substance in the solid form can be dispersed favorably in the oil phase without aggregation, leading to reduction of the feeling of surface-roughness. Furthermore, since the external agent for skin or mucous membrane is the O/W type emulsion with the aqueous phase as the outer phase, the external agent has favorable spreadability, and can be widely and evenly applied to the skin. Furthermore, the external agent for skin of the present invention provides a higher level of feeling of coverage even when the external agent is applied thinly on the skin.

The emulsified state of the oil agent containing the stabilized petrolatum is achieved by the emulsification using the particles of the polycondensation polymer or the vesicles. It should be noted that emulsification of a highly viscous oil agent like petrolatum with surfactants or other agents that have been conventionally used as emulsifiers is impossible.

In addition, unlike emulsification with conventional surfactants, the petrolatum and the emulsion structure stabilized in this way is not destroyed when the external agent for skin or mucous membrane is diluted with water, since the emulsifier particles (the particles of the polycondensation polymer and/or the vesicles) form a separate phase and are not in equilibrium with the free emulsifier. Therefore, such an external agent for skin or mucous membrane can be easily diluted with water, leading to the adjustment of its concentration and viscosity as appropriate. On the other hand, when the petrolatum-containing external agent for skin which is obtained according to the method of, for example, Patent Document 1 is used, the dilution of such a petrolatum-containing external agent with water is impossible.

The polycondensation polymer having a hydroxyl group may be either a natural polymer or a synthetic polymer, and may be selected as appropriate according to the application of the emulsifier. However, natural polymers are preferred because they are excellent in safety and generally inexpensive, and sugar polymers, as described below, are more preferred because of their superior emulsifying function. It should be noted that the particles encompasses a single particle of the polycondensation polymer and a series of the single particles, but does not encompass agglomerates (with a network structure) before forming the single particle.

The sugar polymers are polymers having a glucoside structure such as cellulose and starch. Examples of such a sugar polymer include: those produced by microorganisms using some sugars from among monosaccharides including ribose, xylose, rhamnose, fucose, glucose, mannose, glucuronic acid, and gluconic acid as a constitutive element; natural polymers such as xanthan gum, gum arabic, guar gum, karaya gum, carrageenan, pectin, fucoidan, quince seed gum, trant gum, locust bean gum, galactomannan, curdlan, gellan gum, fucogel, casein, gelatin, starch, collagen, hyaluronic acid, hyaluronic acid derivatives, and Tremella fuciformis polysaccharides; semisynthetic polymers such as methylcellulose, ethylcellulose, methylhydroxypropylcellulose, carboxymethylcellulose, hydroxymethylcellulose, hydroxypropylcellulose, sodium carboxymethylcellulose, alginic acid propylene glycol ester, crystalline cellulose, starch-sodium acrylate graft polymers, and hydrophobically modified hydroxypropylmethylcellulose; synthetic polymers such as polyvinyl alcohol, polyvinylpyrrolidone, carboxyvinyl polymers, polyacrylic acid salts, and polyethylene oxide.

The amphiphilic substance that forms the vesicles is not particularly limited, and examples thereof include poly(oxyethylene) hydrogenated castor oil derivatives represented by the following general formula 1.

General Formula 1

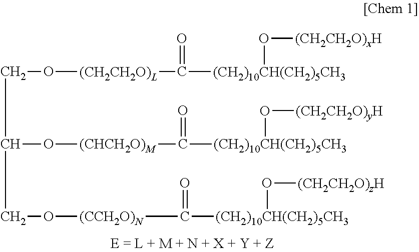

In the formula, E, which represents the average number of moles of ethylene oxide added, represents 3 to 100.

Substances having a hydrophobic group and a hydrophilic group that are bonded via an ester linkage, such as phospholipids and phospholipid derivatives, may be especially employed as the amphiphilic substance.

Among a group of compounds represented by the following general formula 2, DLPC (1,2-Dilauroyl-sn-glycero-3-phospho-rac-1-choline) with a carbon chain length of 12, DMPC (1,2-Dimyristoyl-sn-glycero-3-phospho-rac-1-choline) with a carbon chain length of 14, and DPPC (1,2-Dipalmitoyl-sn-glycero-3-phospho-rac-1-choline) with a carbon chain length of 16 may be employed as the phospholipid.

General Formula 2

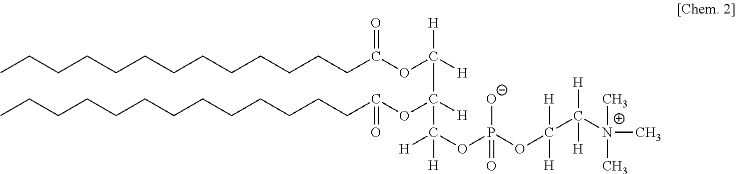

In addition, among a group of compounds represented by the following general formula 3, a Na salt or NH₄ salt of DLPG (1,2-Dilauroyl-sn-glycero-3-phospho-rac-1-glycerol) with a carbon chain length of 12, a Na salt or NH₄ salt of DMPG (1,2-Dimyristoyl-sn-glycero-3-phospho-rac-1-glycerol) with a carbon chain length of 14, and a Na salt or NH₄ salt of DPPG (1,2-Dipalmitoyl-sn-glycero-3-phospho-rac-1-glycerol) with a carbon chain length of 16 may be employed.

General Formula 3

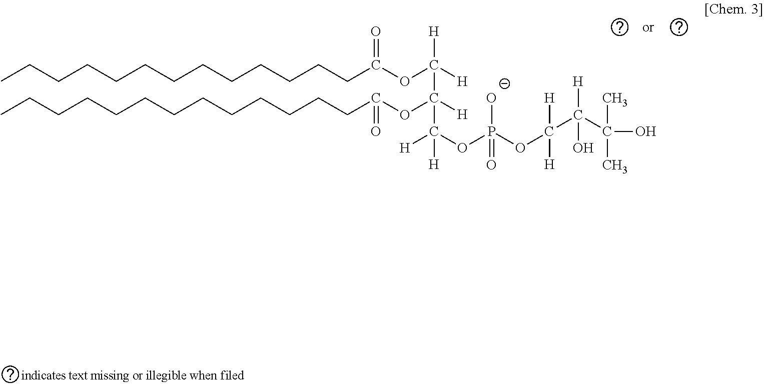

Furthermore, lecithin such as egg yolk lecithin or soybean lecithin may be employed as the phospholipid.

Fatty acid esters may be used as the amphiphilic substance. The fatty acid esters include, for example, glycerin fatty acid esters, polyglycerin fatty acid esters, sucrose fatty acid esters, sorbitan fatty acid esters, propylene glycol fatty acid esters, and the like.

The polyglycerin fatty acid esters are esters of polyglycerin and linear or branched fatty acids, and specific examples thereof include polyglyceryl monopalmitate, polyglyceryl dipalmitate, polyglyceryl tripalmitate, polyglyceryl monostearate, polyglyceryl distearate, polyglyceryl tristearate, polyglyceryl monoisostearate, polyglyceryl diisostearate, polyglyceryl triisostearate, and the like.

The sucrose fatty acid esters include, for example, sucrose myristate ester, sucrose stearate ester, sucrose palmitate ester, sucrose oleate ester, sucrose laurate ester, and the like.

The total amount of the particles of the polycondensation polymer and the vesicles is not particularly limited, but is preferably 0.001% by mass or more, more preferably 0.002% by mass or more, even more preferably 0.005% by mass or more, and particularly preferably 0.01% by mass or more with respect to the total amount of the 01W type emulsion. On the other hand, the total amount of the particles of the polycondensation polymer and the vesicles may be 50% by mass or less, 40% by mass or less, 30% by mass or less, 25% by mass or less, 20% by mass or less, 15% by mass or less, 10% by mass or less with respect to the total amount of the O/W type emulsion.

The particles of the polycondensation polymer and the vesicles have a mean particle diameter of about 8 nm to about 800 nm before the emulsion formation, whereas the particles of the polycondensation polymer and the vesicles have a mean particle diameter of about 8 nm to about 500 nm in the O/W type emulsion structure. Incidentally, only one of the polycondensation polymer particles and the vesicles may be contained, or both of them may be contained. When both of the polycondensation polymer particles and the vesicles are contained, for example, separately emulsified emulsions may be mixed. It should be noted that in the present invention, “mean particle diameter” refers to a value measured by the dynamic light scattering method using a particle size analyzer FPAR (manufactured by Otsuka Electronics Co., Ltd.) and determined by Contin analysis.

### <Oil Phase>

The oil phase consists of petrolatum or is a liquid or a semi-solid including petrolatum with a viscosity of 5000 mPa·s or more at 25° C., and constitutes a part of the inner phase.

### (Petrolatum)

The petrolatum is the main component of the oil phase of the O/W type emulsion structure.

The petrolatum for use in the oil phase of the external agent for skin or mucous membrane is not particularly limited, and, for example, one or more types of commercially available petrolatum such as Sun White P-150, Sun White P-200, and Sun White S-200 (each, from Nikko Rica Corporation), Nomucoat W (from The Nisshin OilliO Group, Ltd.), CROLATUM V (from Croda Japan KK), Penreco Snow, Penreco Ultima, and petrolatum base No. 4 (each, from Penreco), PROTOPET Super White, SONNECONE DM1, SONNECONE CM, MINERAL GELLY #10, MINERAL GELLY #14, MINERAL GELLY #17, SONOJELL #4, and SONOJELL #9 (each from Sonneborn) may be used.

The total amount of the petrolatum with respect to the external agent for skin or mucous membrane may be in an amount of 1% by mass to 80% by mass. In addition, from the viewpoint of obtaining sufficient moisturizing properties of the petrolatum, the total amount of the petrolatum with respect to the external agent for skin or mucous membrane is preferably 2% by mass or more, more preferably 3% by mass or more, even more preferably 5% by mass or more, and particularly preferably 7% by mass or more. When the total amount of the petrolatum is above the required amount, the feeling of coverage can be sufficiently obtained after the coating, whereas the external agent for skin or mucous membrane according to an embodiment of the present invention exhibits low viscosity despite containing such a level of the amount of the petrolatum, and hence excellent feeling during use. From the viewpoint of reduction of the stickiness, the total amount of the petrolatum with respect to the external agent for skin or mucous membrane is preferably 70% by mass or less, more preferably 60% by mass or less, even more preferably less than 50% by mass, and particularly preferably 40% by mass or less. A lower total amount of the petrolatum achieves a lower viscosity of the external agent for skin or mucous membrane, resulting in the formation of an external agent for skin or mucous membrane exhibiting more favorable spreadability. As a result, a lower content of the petrolatum in combination of thinner coating of the external agent for skin or mucous membrane is superior in that the feeling of coverage (moisturizing property) can be sufficiently achieved.

The total amount of the oil phase containing the petrolatum with respect to the external agent for skin or mucous membrane may be in an amount of 1% by mass to 80% by mass. In addition, the total amount of the oil phase containing the petrolatum may be 2% by mass or more, 3% by mass or more, 5% by mass or more, or 7% by mass or more. On the other hand, the total amount of the petrolatum with respect to the external agent for skin or mucous membrane may be 70% by mass or less, 60% by mass or less, less than 50% by mass, or 40% by mass or less.

### <Water-Insoluble Functional Component Phase>

The water-insoluble functional component phase includes the water-insoluble functional component, and constitutes a part of the inner phase.

### (Water-Insoluble Functional Component)

The content of the water-insoluble functional component is not particularly limited, but may be, with respect to the external agent for skin or mucous membrane, in an amount of 0.001% by mass to 50% by mass, and is preferably 0.05% by mass to 40% by mass, and more preferably in an amount of 0.1% by mass to 30% by mass.

### <Aqueous Phase>

The aqueous phase is a medium for dispersing the petrolatum as the oil phase in the O/W type emulsion structure.

The content of the aqueous phase is not particularly limited, but may be, with respect to the external agent for skin or mucous membrane, in an amount of 20% by mass to 99.99% by mass, and is preferably 25% by mass to 98.5% by mass, and more preferably in an amount of 30% by mass to 98% by mass.

It should be noted that the aqueous phase may include, in addition to water, optional components as described later.

The viscosity of the external agent for skin or mucous membrane is not particularly limited, but, for example, is preferably 400000 mPa·s or less, more preferably 200000 mPa·s or less, even more preferably 5000 mPa·s or less, and particularly preferably 3000 mPa·s or less. On the other hand, the viscosity may be, for example, 10 mPa·s or more, 20 mPa·s or more, or 50 mPa·s or more, and further may be 100 mPa·s or more, or 200 mPa·s or more. The “viscosity” as used herein refers to a value measured under the conditions of 25° C. and 1 min using a B-type viscometer, model VSA1, manufactured by Shibaura Systems Co., Ltd. As described above, the present invention can provide an external agent for skin or mucous membrane exhibiting a low viscosity despite containing the petrolatum. It should be noted that the commercially available petrolatum is typically too hard at 25° C. to measure the viscosity of the commercially available petrolatum at 25° C. by the B-type viscometer.

The external agent for skin or mucous membrane of the present invention can easily form an emulsified state by stirring, etc., and the external agent, in the emulsified state, contains the O/W type emulsion structure and stably maintains the emulsified state.

The external agent for skin or mucous membrane of the present invention may take various forms such as a liquid, an emulsion, a cream, a solid, or a gel.

As described above, the O/W emulsion type external agent for skin or mucous membrane that contains a stably emulsified petrolatum without the use of any thickener or emulsification aid can be obtained. Further, such an external agent for skin or mucous membrane of the present invention has the O/W emulsion type structure, and remarkably suppresses the feeling of stickiness due to the petrolatum base and the feeling of surface-roughness due to the functional component, both of which are conventional problems, allowing the coating of the external agent for skin or mucous membrane without incompatibility with the skin or a sense of discomfort to the skin.

«External agent for Skin or Mucous membrane according to Second Aspect»

An external agent for skin or mucous membrane according to a second aspect is characterized by including an oil phase, as an inner phase, that consists of petrolatum and a water-insoluble functional component or is a liquid or a semi-solid including petrolatum and a water-insoluble functional component with a viscosity of 5000 mPa·s or more at 25° C., and an aqueous phase as an outer phase, in which particles of a polycondensation polymer having a hydroxyl group and/or vesicles formed of an amphiphilic substance are present at the interface between the oil phase and the aqueous phase.

More specifically, in the external agent for skin or mucous membrane according to the first aspect, the petrolatum and the water-insoluble functional component are each placed in separate inner phases (emulsion particles), whereas in this external agent for skin or mucous membrane according to the second aspect, the petrolatum and the water-insoluble functional component are placed in identical inner phases (emulsion particles). This external agent for skin or mucous membrane according to the second aspect also reduces a feeling of stickiness and a feeling of surface-roughness on the skin or mucous membrane, exhibits favorable spreadability, and provides a high level of feeling of coverage, similarly to the external agent for skin or mucous membrane according to the first aspect.

The external agent for skin or mucous membrane according to the second aspect is similar to the external agent for skin or mucous membrane according to the first aspect except that the oil phase further contains the water-insoluble functional component, and that the water-insoluble functional component phase is not an essential component. Therefore, only the oil phase will be described in this section.

### <Oil Phase>

In the external agent for skin or mucous membrane according to the second aspect, the oil phase consists of the petrolatum and the water-insoluble functional component or is a liquid or a semi-solid including the petrolatum and the water-insoluble functional component with a viscosity of 5000 mPa·s or more at 25° C., and constitutes a part of the inner phase.

The type and content of the petrolatum and the water-insoluble functional component are also similar to those for the external agent for skin or mucous membrane according to the first aspect.

The external agents for skin or mucous membrane according to the first and second aspects as described above may contain various types of components other than the functional component in the oil phase, and may contain the functional component or various types of other components in the aqueous phase.

### «External Agent for Skin or Mucous Membrane According to Third Aspect»

An external agent for skin or mucous membrane according to a third aspect is characterized by including an oil phase, as an inner phase, that consists of petrolatum or is a liquid or a semi-solid including petrolatum with a viscosity of 5000 mPa·s or more at 25° C., and an aqueous phase, as an outer phase, including a water-soluble functional component, in which particles of a polycondensation polymer having a hydroxyl group and/or vesicles formed of an amphiphilic substance are present at the interface between the oil phase and the aqueous phase.

More specifically, the external agents for skin or mucous membrane according to the first and second aspects as described above use a water-insoluble ingredient as the functional component, whereas this external agent for skin or mucous membrane according to the third aspect uses a water-soluble ingredient as the functional component. This external agent for skin or mucous membrane according to a third aspect also reduces a feeling of stickiness on the skin or mucous membrane due to the petrolatum, similarly to the external agent for skin or mucous membrane according to the first aspect. As described above, for example, the external agent described in Patent Document 1 lacks water, and a water-soluble functional component is present in the external agent without dissolution in the external agent, which causes a feeling of surface-roughness. In contrast, this external agent for skin or mucous membrane according to the third aspect contains water as the outer phase, and the water-soluble functional component is contained in the aqueous phase. Thus, the water-soluble functional component is dissolved, leading to the suppression of the feeling of surface-roughness. Further, the external agent for skin or mucous membrane according to the third aspect exhibits favorable spreadability, and provides a higher level of feeling of coverage.

The external agent for skin or mucous membrane according to the third aspect is similar to the external agent for skin or mucous membrane according to the first aspect except that the aqueous phase further contains the water-soluble functional component, and that the water-insoluble functional component phase is not an essential component. Therefore, only the aqueous phase will be described in this section.

### <Aqueous Phase>

In the external agent for skin or mucous membrane according to a third aspect, the aqueous phase includes the water-soluble functional component, and constitutes the outer phase.

### (Water-Soluble Functional Component)

The water-soluble functional component is placed in the aqueous phase in a dissolved state.

The content of the water-soluble functional component is not particularly limited, but may be, with respect to the external agent for skin or mucous membrane, in an amount of 0.001% by mass to 50% by mass, and is preferably 0.001% by mass to 45% by mass, and more preferably in an amount of 0.001% by mass to 40% by mass.

The external agent for skin or mucous membrane according to the third aspect may contain various types of components other than the functional component in the aqueous phase, and may contain the functional component or various types of other components in the oil phase.

The properties of the external agents for skin or mucous membrane according to the first to third aspects as described above are not particularly limited, and can be used as an ointment.

It should be noted that the coating target of the external agents for skin or mucous membrane according to the first to third aspects as described above is not limited to humans, and may be animals (dogs, cats, cows, horses, birds, etc.).

«Production Method of External Agents for Skin or Mucous Membrane according to First and Third Aspects»

The production method of the external agents for skin or mucous membrane according to the first and third aspects as described above is described. First, an oil consisting of or including the petrolatum heated to the melting point of the oil or higher to melt is added dropwise to water in which the particles of the polycondensation polymer having the hydroxyl group and/or the vesicles formed of the amphiphilic substance is dispersed, followed by mixing with stirring the water. This procedure gives a basic ingredient emulsion composition containing an oil phase, as an inner phase, that consists of petrolatum or is a liquid or a semi-solid including petrolatum with a viscosity of 5000 mPa·s or more at 25° C., an aqueous phase as an outer phase, and particles of a polycondensation polymer having a hydroxyl group and/or vesicles formed of an amphiphilic substance, and the basic ingredient emulsion composition has a structure in which a part of the particles and/or the vesicles are present at the interface between the oil phase and the aqueous phase.

Incidentally, the method for producing water containing dispersed closed vesicles formed by an amphiphilic substance forming the closed vesicles and/or dispersed single particles of a polycondensation polymer having a hydroxyl group is omitted because the method is conventionally known, for example, in Japanese Patent No. 3855203. The amount of each blended component as well as optional components are as described above.

Then, the water-insoluble functional component is added to the resulting basic ingredient emulsion composition. This results in, separately from the oil phase formed by the petrolatum, the emulsification of the water-insoluble functional component by the polycondensation polymer particles and/or the vesicles present in excess in the system, and the dispersion of the water-insoluble functional component in the water as the outer phase, to yield the external agent for skin or mucous membrane according to the first aspect.

On the other hand, when the water-soluble functional component is added to the resulting basic ingredient emulsion composition, this water-soluble functional component is dissolved in water, to yield the external agent for skin or mucous membrane according to the third aspect.

«Production Method of External Agent for Skin or Mucous Membrane according to Second Aspect»

The external agent for skin or mucous membrane according to the second aspect is obtained by mixing a water-insoluble functional component with the oil consisting of or including the petrolatum followed by emulsification in the course of the production of the basic ingredient emulsion composition as described above.

### «Base for External Agent for Skin or Mucous Membrane»

As described above, the external agents for skin or mucous membrane according to the first and third aspects can be easily obtained by adding the water-soluble functional component and/or the water-soluble functional component to the basic ingredient emulsion composition containing the oil phase, as the inner phase, that consists of petrolatum or is a liquid or a semi-solid including the petrolatum with a viscosity of 5000 mPa·s or more at 25° C., the aqueous phase as the outer phase, and the particles of the polycondensation polymer having the hydroxyl group and/or the vesicles formed of the amphiphilic substance, in which a part of the particles and/or the vesicles are present at the interface between the oil phase and the aqueous phase. In other words, the basic ingredient emulsion composition as described above may be referred to as a base for the external agents for skin or mucous membrane according to the first and third aspects.

According to such a base for an external agent for skin or mucous membrane, when the water-soluble functional component is added, the water-soluble functional component is dissolved in the aqueous phase (outer phase) of the O/W type emulsion, and on the other hand, when the water-insoluble functional component is added, the water-insoluble functional component coexists in the liquid separately from the oil phase (inner phase) associated with the petrolatum. Thus, a stable emulsion composition can be formed for any kind of substance added.

### EXAMPLES

In the following, the present invention will be described in more detail with reference to Examples and Comparative Examples of the present invention, but the present invention is not limited the following Examples.

### (Base 1)

A dispersion prepared by adding 30 g of ion-exchanged water to 50 g of a 0.5% by mass dispersion of stearoxy hydroxypropylmethyl cellulose (SANGELOSE 90L, Daido Chemical Corporation) was heated at 90° C. with stirring, and thereto 20 g of white petrolatum heated at 90° C. was added dropwise. After the dropwise addition of the total amount of the white petrolatum, the heating was maintained for another 5 minutes with stirring. Then, the mixture was cooled with stirring. The resulting liquid was a white emulsion. The mean particle diameter of the obtained emulsion was 4 to 5 μm.

### (Base 2)

An emulsion was prepared by using the same composition as that of the base 1 and adjusting the stirring speed of the homomixer and other factors such that the emulsion had a mean particle diameter of 20 μm. The emulsion was then cooled with stirring, and the resulting emulsion was white in color.

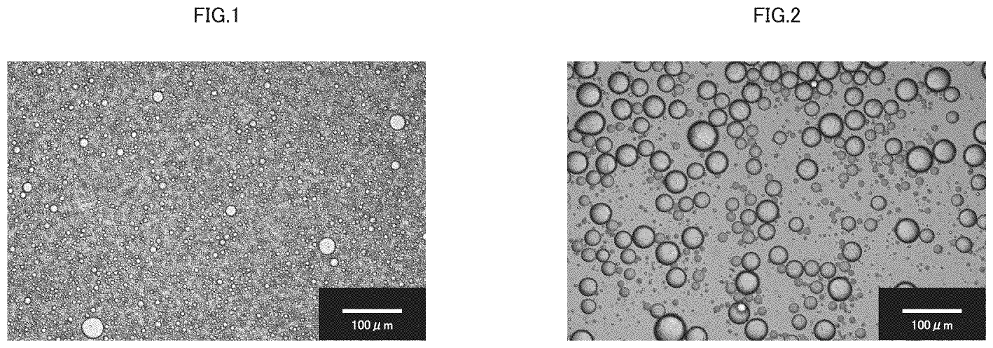

Microscopic observation was performed on the bases obtained as described above. FIG. 1 shows a microscopy image of the base 1. FIG. 2 shows a microscopy image of the base 2. In FIG. 1 and FIG. 2, a large number of spherical particles were observed in the aqueous phase. It was found that these spherical particles were emulsion particles, and the oil phase of the petrolatum was dispersed in the aqueous phase in the bases.

Further, the bases 1 and 2 were transferred to a glass bottle, and the emulsified state of each base was observed by a microscope after the bases were left to stand for 24 h. This observation revealed that a stable emulsified state was maintained.

### <Evaluation of Emulsified State>

### Example 1

The base 1 and allantoin powder were mixed such that 99.6% by mass of the base 1 and 0.4% by mass of the allantoin powder were contained. The resulting sample was a white emulsion.

It should be noted that the allantoin has tissue-repair-activating action (action to repair and activate the skin tissue), anti-irritant action, anti-inflammatory soothing action, and anti-allergic action, and the like, and is effective for rough skin and acne.

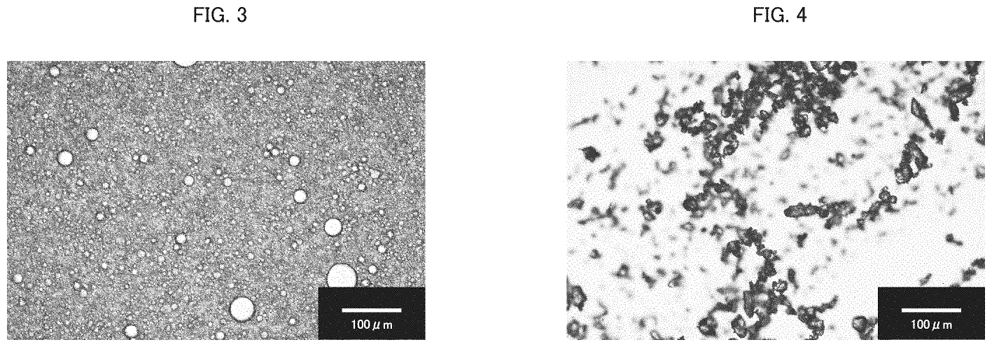

FIG. 3 shows a microscopy image of the sample obtained in Example 1. FIG. 4 shows a microscopy image of the allantoin powder. In the following, explanation is made with reference to FIGS. 3, 4 and 1. It should be noted that FIGS. 3, 4 and 1 are microscopy images of the same magnification.

The sample obtained in Example 1 (see FIG. 3) was obtained by adding the allantoin powder (see FIG. 4) to the base 1 (see FIG. 1), and there was no significant difference in the microscopy images from the base 1 (see FIG. 1), and no powdered allantoin was observed, whereas irregularly powdered allantoin was observed in FIG. 4. The allantoin is a water-soluble substance, and dissolution of the allantoin in the aqueous phase as the outer phase probably leads to the above observation. It was found that the dissolution of a water-soluble substance like allantoin in the aqueous phase as the outer phase had no adverse effect on the emulsified state.

### Example 2

The base 1 and cerebroside powder were mixed such that 90.0% by mass of the base 1 and 10.0% by mass of the cerebroside powder were contained. The resulting sample was a white emulsion.

It should be noted that the cerebroside is a ceramide derived from animals such as horses, and is a component close to human skin. The cerebroside has an excellent moisturizing effect and blends well with the skin.

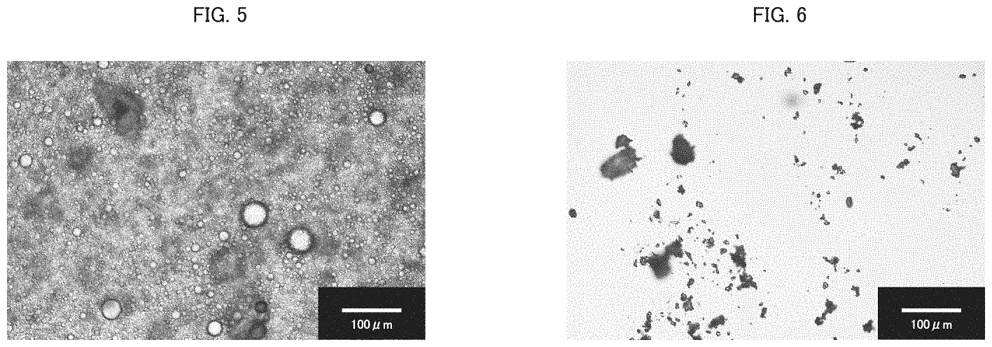

FIG. 5 shows a microscopy image of the sample obtained in Example 2. FIG. 6 shows a microscopy image of the cerebroside powder. In the following, explanation is made with reference to FIGS. 5, 6 and 1. It should be noted that FIGS. 5, 6 and 1 are microscopy images of the same magnification.

The sample obtained in Example 2 (see FIG. 5) was obtained by adding the cerebroside powder (see FIG. 6) to base 1 (see FIG. 1), but unlike the base 1 (see FIG. 1), an irregularly powdered substance differing in contrast from the spherical emulsion particles containing the petrolatum was observed in the microscopy image in addition to the spherical emulsion particles. The irregularly powdered substance is generally similar in morphology to the cerebroside powder prior to the addition, and it was found that in the sample obtained in Example 2, the irregularly powdered cerebroside observed in FIG. 6 was present in the aqueous phase as the outer phase in a dispersed state. The cerebroside is a water-insoluble substance, and it is considered that the cerebroside is not dissolved in the aqueous phase as the outer phase and emulsified and dispersed by the stearoxy hydroxypropylmethyl cellulose contained in the base 1.

### Example 3

The base 2 and an aqueous dispersion of 32% by mass titanium oxide (primary particle diameter: 12 to 15 nm) were mixed such that 90.0% by mass of the base 2 and 10.0% by mass of the aqueous dispersion were contained. The resulting sample was a white emulsion.

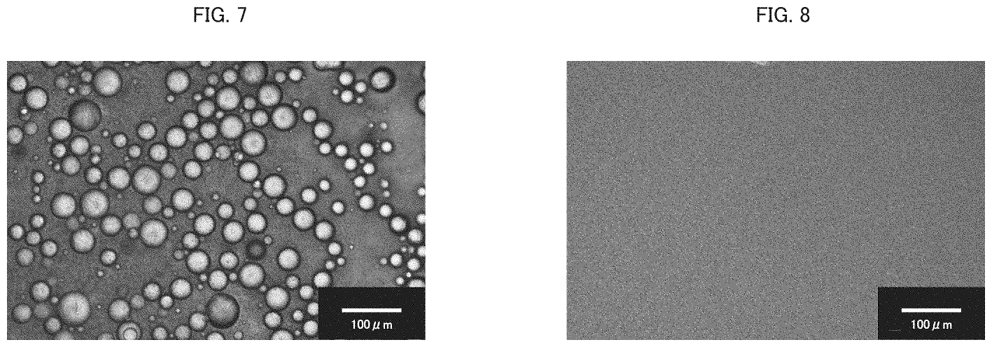

FIG. 7 shows a microscopy image of the sample obtained in Example 3. FIG. 8 shows a microscopy image of the aqueous dispersion of 32% by mass titanium oxide. In the following, explanation is made with reference to FIGS. 7, 8 and 2. It should be noted that FIGS. 7, 8 and 2 are microscopy images of the same magnification.

The sample obtained in Example 3 (see FIG. 7) was obtained by adding the aqueous dispersion of 32% by mass titanium oxide (see FIG. 8) to the base 2 (see FIG. 2). This titanium oxide has a primary particle diameter of 12 to 15 nm as described above, and has an extremely fine size relative to the magnification of the microscope even in view of aggregation of the titanium oxide, and thus observation of a single particle of the titanium oxide is impossible in FIG. 8. Nonetheless, when the titanium oxide particles are dispersed, the contrast of the image is darker than when the titanium oxide particles are not dispersed (see the outer phase in FIG. 2). In the sample obtained in Example 3 (see FIG. 7), there is no significant difference between the sample obtained in Example 3 (see FIG. 5) and the base 2 (see FIG. 2) in terms of the contrast of the image of the inner phase consisting of the petrolatum in the microscopy image as compared with the base 2 (see FIG. 2). On the other hand, the contrast of the images of areas other than the inner phase consisting of the petrolatum is darker. Although individual emulsions of the titanium oxide could not be confirmed due to limited observation magnification of the microscope, it was confirmed that in the sample obtained in Example 3, the titanium oxide is at least not present in identical emulsions (inner phase) to those of the petrolatum. However, it has been confirmed that when the particles of stearoxy hydroxypropylmethyl cellulose is added to the aqueous dispersion of titanium oxide, the titanium oxide is emulsified by these particles. Therefore, it is considered that the titanium oxide forms emulsions (inner phase) separate from those of the petrolatum, and is dispersed in water.

### Example 4

The base 1 and a silicone oil dispersion of 29% by mass titanium oxide (primary particle diameter: 12 to 15 nm) were mixed such that 90.0% by mass of the base 1 and 10.0% by mass of the silicone oil dispersion were contained. The resulting sample was a white emulsion.

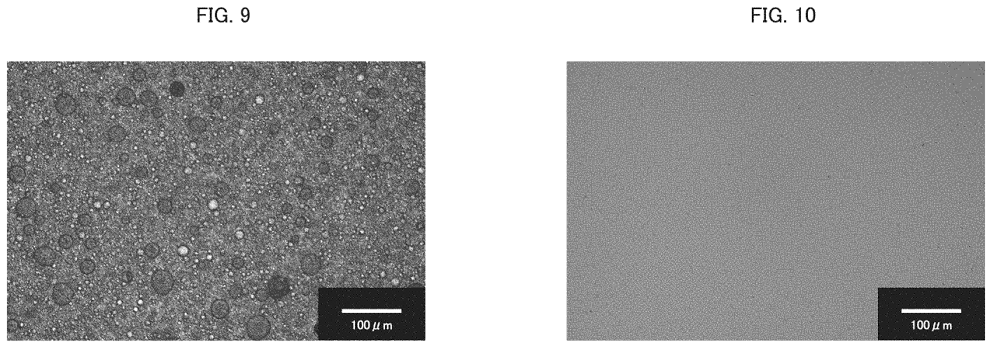

FIG. 9 shows a microscopy image of the sample obtained in Example 4. FIG. 10 shows a microscopy image of the silicone oil dispersion of 29% by mass titanium oxide. In the following, explanation is made with reference to FIGS. 9, 10 and 1. It should be noted that FIGS. 9, 10 and 1 are microscopy images of the same magnification.

The sample obtained in Example 4 (see FIG. 9) was obtained by adding the silicone oil dispersion of 29% by mass titanium oxide (see FIG. 10) to the base 1 (see FIG. 1). Although this titanium oxide is similar to the titanium oxide used in Example 3 and observation of a single particle of the titanium oxide is impossible in FIG. 10, the contrast of the image is darker. In the sample obtained in Example 4 (see FIG. 9), spherical emulsion particles exhibiting darker contrast than the spherical emulsion particles exhibiting lighter contrast were observed in addition to the spherical emulsion particles exhibiting lighter contrast observed in the base 1 (see FIG. 1), as compared with the base 1 (see FIG. 1). Therefore, it was confirmed that in the sample obtained in Example 4, emulsions containing the silicone oil and titanium oxide were present in addition to the emulsions of the petrolatum.

From the results described above, it was found that the use of specific polycondensation polymers and vesicles allows for the production of cosmetics and pharmaceuticals that contain petrolatum as a base.

### <Sensory Evaluation>

### Example 5

The base 1 and allantoin powder were mixed such that 99.9% by mass of the base 1 and 0.1% by mass of the allantoin powder were contained. The resulting sample was a white emulsion.

Sensory evaluation of the sample obtained in Example 5 and the Japanese Pharmacopoeia white petrolatum as a comparative sample was conducted by 10 female panelists in their 20s to 40s. Specifically, the sample obtained in Example 1 was coated to the left forearm from the wrist to the upper arm, and the Japanese Pharmacopoeia white petrolatum was coated to the right forearm from the wrist to the upper arm, each in an amount of 1 FTU (the amount that would fit on the first joint of the index finger). Properties “good spreadability”, “quick blending”, “smoothness”, “stickiness and dryness”, and “feeling of coverage” of each sample were evaluated on a 5-point scale of “very good” (2), “good” (1), “neither good nor bad” (0), “bad” (−1), and “very bad” (−2).

Tables 1 to 5 show the results of the sensory evaluation of the sample obtained in Example 5 and the Japanese Pharmacopoeia white petrolatum in terms of “good spreadability” (Table 1), “quick blending” (Table 2), “smoothness” (Table 3), “stickiness and dryness” (Table 4), and “feeling of coverage” (Table 5), respectively. In addition, Table 6 shows the total of the numerical values of the sensory evaluation results of the 10 panelists (the numerical values of 2 to −2 as described above). As described above, the sample obtained in Example 5 was found to exhibit a higher level of feeling during use than the conventional petrolatum in terms of “good spreadability”, “quick blending”, “smoothness”, and “stickiness and dryness”, while maintaining the “feeling of coverage”.

TABLE 1
(Good spreadability)

| | Sample | Japanese |
|---|---|---|
| | obtained | Pharmacopoeia |
| | in | white |
| Panelist | Example 5 | petrolatum |
| 1 | 2 | −2 |
| 2 | 2 | −2 |
| 3 | 2 | −1 |
| 4 | 2 | −2 |
| 5 | 2 | −2 |
| 6 | 2 | −2 |
| 7 | 2 | −2 |
| 8 | 2 | −2 |
| 9 | 2 | −2 |
| 10 | 2 | −2 |

TABLE 2
(Quick blending)

| | Sample | Japanese |
|---|---|---|
| | obtained in | Pharmacopoeia |
| Panelist | Example 5 | white petrolatum |
| 1 | 2 | −2 |
| 2 | 2 | −2 |
| 3 | 2 | −2 |
| 4 | 2 | −2 |
| 5 | 2 | −2 |
| 6 | 2 | −2 |
| 7 | 2 | −2 |
| 8 | 2 | −2 |
| 9 | 2 | −2 |
| 10 | 2 | −2 |

TABLE 3
(Smoothness)

| | Sample | Japanese |
|---|---|---|
| | obtained in | Pharmacopoeia |
| Panelist | Example 5 | white petrolatum |
| 1 | 2 | −2 |
| 2 | 2 | −1 |
| 3 | 2 | −2 |
| 4 | 2 | −2 |
| 5 | 2 | −2 |
| 6 | 2 | −2 |
| 7 | 2 | −2 |
| 8 | 2 | −1 |
| 9 | 2 | −2 |
| 10 | 2 | −2 |

TABLE 4
(Stickiness and dryness)

| | Sample | Japanese |
|---|---|---|
| | obtained in | Pharmacopoeia |
| Panelist | Example 5 | white petrolatum |
| 1 | 1 | −1 |
| 2 | 1 | −2 |
| 3 | 1 | −1 |
| 4 | 2 | −2 |
| 5 | 2 | −1 |
| 6 | 2 | −2 |
| 7 | 2 | −2 |
| 8 | 1 | −1 |
| 9 | 2 | −2 |
| 10 | 1 | −2 |

TABLE 5
(Feeling of coverage)

| | Sample | Japanese |
|---|---|---|
| | obtained in | Pharmacopoeia |
| Panelist | Example 5 | white petrolatum |
| 1 | 2 | 2 |
| 2 | 1 | 2 |
| 3 | 1 | 2 |
| 4 | 2 | 2 |
| 5 | 1 | 2 |
| 6 | 2 | 2 |
| 7 | 2 | 2 |
| 8 | 1 | 2 |
| 9 | 2 | 2 |
| 10 | 1 | 2 |

TABLE 6

| | Sample obtained | Japanese Pharmacopoeia |
|---|---|---|
| | in Example 5 | white petrolatum |
| Good spreadability | 20 | −19 |
| Quick blending | 20 | −20 |
| Smoothness | 20 | −18 |
| Stickiness and dryness | 15 | −16 |
| Feeling of coverage | 15 | 20 |

### Example 6

The base 1 and triamcinolone acetonide powder were mixed such that 99.9% by mass of the base 1 and 0.1% by mass of the triamcinolone acetonide powder were contained. The resulting sample was a white emulsion.

Sensory evaluation of the sample obtained in Example 6 and a commercially available triamcinolone acetonide 0.1% ointment (Ledercort(registered) ointment) as a comparative sample was conducted in a similar manner to Example 5. It should be noted that the triamcinolone acetonide 0.1% ointment is prepared by using petrolatum as a base and kneading triamcinolone acetonide as an active ingredient and various types of additives.

Tables 7 to 11 show the results of the sensory evaluation of the sample obtained in Example 6 and the triamcinolone acetonide 0.1% ointment in terms of “good spreadability” (Table 7), “quick blending” (Table 8), “smoothness” (Table 9), “stickiness and dryness” (Table 10), and “feeling of coverage” (Table 11), respectively. In addition, Table 12 shows the total of the numerical values of the sensory evaluation results of the 10 panelists (the numerical values of 2 to −2 as described above). As described above, the sample obtained in Example 6 was found to exhibit a higher level of feeling during use than the commercial ointment in terms of “good spreadability”, “quick blending”, “smoothness”, and “stickiness and dryness”, while having the “feeling of coverage”.

TABLE 7
(Good spreadability)

| | Sample | Triamcinolone |
|---|---|---|
| | obtained | acetonide |
| Panelist | in Example 6 | 0.1% ointment |
| 1 | 2 | −2 |
| 2 | 2 | −2 |
| 3 | 2 | −1 |
| 4 | 2 | −2 |
| 5 | 2 | −2 |
| 6 | 2 | −2 |
| 7 | 2 | −2 |
| 8 | 2 | −2 |
| 9 | 2 | −2 |
| 10 | 2 | −2 |

TABLE 8
(Quick blending)

| | Sample | Triamcinolone |
|---|---|---|
| | obtained in | acetonide |
| Panelist | Example 6 | 0.1% ointment |
| 1 | 2 | −2 |
| 2 | 2 | −1 |
| 3 | 2 | −2 |
| 4 | 2 | −2 |
| 5 | 2 | −2 |
| 6 | 2 | −2 |
| 7 | 2 | −2 |
| 8 | 2 | −2 |
| 9 | 2 | −2 |
| 10 | 2 | −2 |

TABLE 9
(Smoothness)

| | Sample | Triamcinolone |
|---|---|---|
| | obtained in | acetonide |
| Panelist | Example 6 | 0.1% ointment |
| 1 | 2 | −2 |
| 2 | 2 | −1 |
| 3 | 2 | −2 |
| 4 | 2 | −2 |
| 5 | 2 | −2 |
| 6 | 2 | −2 |
| 7 | 2 | −2 |
| 8 | 2 | −1 |
| 9 | 2 | −2 |
| 10 | 2 | −2 |

TABLE 10
(Stickiness and dryness)

| | Sample | Triamcinolone |
|---|---|---|
| | obtained in | acetonide |
| Panelist | Example 6 | 0.1% ointment |
| 1 | 2 | −2 |
| 2 | 1 | −2 |
| 3 | 2 | −1 |
| 4 | 2 | −2 |
| 5 | 1 | −1 |
| 6 | 2 | −2 |
| 7 | 2 | −2 |
| 8 | 1 | −2 |
| 9 | 2 | −2 |
| 10 | 1 | −2 |

TABLE 11
(Feeling of coverage)

| | Sample | Triamcinolone |
|---|---|---|
| | obtained in | acetonide |
| Panelist | Example 6 | 0.1% ointment |
| 1 | 2 | 2 |
| 2 | 1 | 2 |
| 3 | 1 | 2 |
| 4 | 2 | 2 |
| 5 | 1 | 2 |
| 6 | 2 | 2 |
| 7 | 2 | 2 |
| 8 | 1 | 2 |
| 9 | 2 | 2 |
| 10 | 1 | 2 |

TABLE 12

| | Sample | Triamcinolone |
|---|---|---|
| | obtained in | acetonide |
| | Example 6 | 0.1% ointment |
| Good spreadability | 20 | −19 |
| Quick blending | 20 | −19 |
| Smoothness | 20 | −18 |
| Stickiness and dryness | 16 | −18 |
| Feeling of coverage | 15 | 20 |

### Example 7

The base 1 and betamethasone valerate were mixed such that 99.9% by mass of the base 1 and 0.12% by mass of betamethasone valerate were contained. The resulting sample was a white emulsion.

Sensory evaluation of the sample obtained in Example 7 and a commercially available betamethasone valerate 0.12% ointment (Rinderon(registered)-V ointment) as a comparative sample was conducted in a similar manner to Example 5. It should be noted that the betamethasone valerate 0.12% ointment is prepared by using petrolatum and liquid paraffin as a base, and kneading betamethasone valerate as an active ingredient.

Tables 13 to 17 show the results of the sensory evaluation of the sample obtained in Example 7 and the commercially available betamethasone valerate 0.12% ointment in terms of “good spreadability” (Table 13), “quick blending” (Table 14), “smoothness” (Table 15), “stickiness and dryness” (Table 16), and “feeling of coverage” (Table 17), respectively. In addition, Table 18 shows the total of the numerical values of the sensory evaluation results of the 10 panelists (the numerical values of 2 to −2 as described above). As described above, the sample obtained in Example 7 was found to exhibit a higher level of feeling during use than the conventional petrolatum in terms of “good spreadability”, “quick blending”, “smoothness”, and “stickiness and dryness”, while having the “feeling of coverage”.

TABLE 13
(Good spreadability)

| | Sample | Betamethasone |
|---|---|---|
| | obtained in | valerate 0.12% |
| Panelist | Example 7 | ointment |
| 1 | 2 | −2 |
| 2 | 2 | −2 |
| 3 | 2 | −1 |
| 4 | 2 | −2 |
| 5 | 2 | −2 |
| 6 | 2 | −2 |
| 7 | 2 | −2 |
| 8 | 2 | −2 |
| 9 | 2 | −2 |
| 10 | 2 | −2 |

TABLE 14
(Quick blending)

| | Sample | Betamethasone |
|---|---|---|
| | obtained in | valerate 0.12% |
| Panelist | Example 7 | ointment |
| 1 | 2 | −2 |
| 2 | 2 | −2 |
| 3 | 2 | −2 |
| 4 | 2 | −2 |
| 5 | 2 | −2 |
| 6 | 2 | −2 |
| 7 | 2 | −2 |
| 8 | 2 | −1 |
| 9 | 2 | −2 |
| 10 | 2 | −2 |

TABLE 15
(Smoothness)

| | Sample | Betamethasone |
|---|---|---|
| | obtained in | valerate 0.12% |
| Panelist | Example 7 | ointment |
| 1 | 2 | −2 |
| 2 | 2 | −1 |
| 3 | 2 | −2 |
| 4 | 2 | −2 |
| 5 | 2 | −1 |
| 6 | 2 | −2 |
| 7 | 2 | −2 |
| 8 | 2 | −1 |
| 9 | 2 | −2 |
| 10 | 2 | −2 |

TABLE 16
(Stickiness and dryness)

| | Sample | Betamethasone |
|---|---|---|
| | obtained in | valerate 0.12% |
| Panelist | Example 7 | ointment |
| 1 | 2 | −2 |
| 2 | 2 | −2 |
| 3 | 1 | −2 |
| 4 | 1 | −2 |
| 5 | 1 | −2 |
| 6 | 2 | −2 |
| 7 | 2 | −1 |
| 8 | 2 | −2 |
| 9 | 2 | −2 |
| 10 | 1 | −2 |

TABLE 17
(Feeling of coverage)

| | Sample | Betamethasone |
|---|---|---|
| | obtained in | valerate 0.12% |
| Panelist | Example 7 | ointment |
| 1 | 1 | 2 |
| 2 | 2 | 2 |
| 3 | 1 | 2 |
| 4 | 2 | 2 |
| 5 | 2 | 2 |
| 6 | 2 | 2 |
| 7 | 2 | 2 |
| 8 | 1 | 2 |
| 9 | 2 | 2 |
| 10 | 2 | 2 |

TABLE 18

| | Sample | Betamethasone |
|---|---|---|
| | obtained in | valerate |
| | Example 7 | 0.12% ointment |
| Good spreadability | 20 | −19 |
| Quick blending | 20 | −19 |
| Smoothness | 20 | −17 |
| Stickiness and dryness | 16 | −19 |
| Feeling of coverage | 17 | 20 |

### Example 8

The base 1 and titanium oxide were mixed such that 93.4% by mass of the base 1 and 6.4% by mass of titanium oxide were contained, and the mixture achieved SPF 24, and PA++. The resulting sample was a white emulsion.

Sensory evaluation of the sample obtained in Example 8 and the Japanese Pharmacopoeia white petrolatum as a comparative sample was conducted in a similar manner to Example 5.

Tables 19 to 23 show the results of the sensory evaluation of the sample obtained in Example 8 and the Japanese Pharmacopoeia white petrolatum in terms of “good spreadability” (Table 19), “quick blending” (Table 20), “smoothness” (Table 21), “stickiness and dryness” (Table 22), and “feeling of coverage” (Table 23), respectively. In addition, Table 24 shows the total of the numerical values of the sensory evaluation results of the 10 panelists (the numerical values of 2 to −2 as described above). As described above, the sample obtained in Example 8 was found to exhibit a higher level of feeling during use than the conventional petrolatum in terms of “good spreadability”, “quick blending”, “smoothness”, and “stickiness and dryness”, while maintaining the “feeling of coverage”.

TABLE 19
(Good spreadability)

| | Sample | Japanese |
|---|---|---|
| | obtained | Pharmacopoeia |
| | in | white |
| Panelist | Example 8 | petrolatum |
| 1 | 1 | −2 |
| 2 | 2 | −2 |
| 3 | 1 | −1 |
| 4 | 1 | −2 |
| 5 | 1 | −2 |
| 6 | 2 | −2 |
| 7 | 1 | −2 |
| 8 | 1 | −2 |
| 9 | 2 | −2 |
| 10 | 1 | −2 |

TABLE 20
(Quick blending)

| | Sample | Japanese |
|---|---|---|
| | obtained | Pharmacopoeia |
| | in | white |
| Panelist | Example 8 | petrolatum |
| 1 | 1 | −2 |
| 2 | 1 | −2 |
| 3 | 2 | −2 |
| 4 | 2 | −2 |
| 5 | 2 | −2 |
| 6 | 1 | −2 |
| 7 | 2 | −2 |
| 8 | 1 | −2 |
| 9 | 2 | −2 |
| 10 | 2 | −2 |

Table 21
(Smoothness)

| | Sample | Japanese |
|---|---|---|
| | obtained | Pharmacopoeia |
| | in | white |
| Panelist | Example 8 | petrolatum |
| 1 | 1 | −2 |
| 2 | 1 | −1 |
| 3 | 1 | −2 |
| 4 | 1 | −2 |
| 5 | 2 | −2 |
| 6 | 1 | −2 |
| 7 | 1 | −2 |
| 8 | 1 | −1 |
| 9 | 1 | −2 |
| 10 | 1 | −2 |

TABLE 22
(Stickiness and dryness)

| | Sample | Japanese |
|---|---|---|
| | obtained | Pharmacopoeia |
| | in | white |
| Panelist | Example 8 | petrolatum |
| 1 | 1 | −2 |
| 2 | 1 | −2 |
| 3 | 1 | −1 |
| 4 | 2 | −2 |
| 5 | 1 | −2 |
| 6 | 2 | −2 |
| 7 | 1 | −2 |
| 8 | 1 | −1 |
| 9 | 1 | −2 |
| 10 | 1 | −2 |

TABLE 23
(Feeling of coverage)

| | Sample | Japanese |
|---|---|---|
| | obtained | Pharmacopoeia |
| | in | white |
| Panelist | Example 8 | petrolatum |
| 1 | 2 | 2 |
| 2 | 2 | 2 |
| 3 | 1 | 2 |
| 4 | 1 | 2 |
| 5 | 1 | 2 |
| 6 | 2 | 2 |
| 7 | 1 | 2 |
| 8 | 1 | 2 |
| 9 | 2 | 2 |
| 10 | 1 | 2 |

TABLE 24

| | Sample | Japanese |
|---|---|---|
| | obtained in | Pharmacopoeia |
| | Example 8 | white petrolatum |
| Good spreadability | 13 | −19 |
| Quick blending | 16 | −20 |
| Smoothness | 11 | −18 |
| Stickiness and dryness | 12 | −18 |
| Feeling of coverage | 14 | 20 |

1. An external agent for skin or mucous membrane, comprising:
an oil phase, as an inner phase, that consists of petrolatum or is a liquid or a semi-solid comprising petrolatum with a viscosity of 5000 mPa·s or more at 25° C., and a water-insoluble functional component phase, as an inner phase, comprising a water-insoluble functional component, and an aqueous phase as an outer phase,
wherein particles of a polycondensation polymer having a hydroxyl group and/or vesicles formed of an amphiphilic substance are present at an interface between the oil phase and the aqueous phase, and at an interface between the water-insoluble functional component phase and the aqueous phase.

2. An external agent for skin or mucous membrane, comprising:
an oil phase, as an inner phase, that consists of petrolatum and a water-insoluble functional component or is a liquid or a semi-solid comprising petrolatum and a water-insoluble functional component with a viscosity of 5000 mPa·s or more at 25° C., and an aqueous phase as an outer phase,
wherein particles of a polycondensation polymer having a hydroxyl group and/or vesicles formed of an amphiphilic substance are present at an interface between the oil phase and the aqueous phase.

3. The external agent for skin or mucous membrane according to claim 1, wherein a content of the water-insoluble functional component with respect to a total amount of the external agent for skin is 50% by mass or less.

4. The external agent for skin or mucous membrane according to claim 1, wherein the water-insoluble functional component is a functional component of cosmetics and/or a functional component of pharmaceuticals.

5. An external agent for skin or mucous membrane, comprising:
an oil phase, as an inner phase, that consists of petrolatum or is a liquid or a semi-solid comprising petrolatum with a viscosity of 5000 mPa·s or more at 25° C., and an aqueous phase, as an outer phase, comprising a water-soluble functional component,
wherein particles of a polycondensation polymer having a hydroxyl group and/or vesicles formed of an amphiphilic substance are present at an interface between the oil phase and the aqueous phase.

6. The external agent for skin or mucous membrane according to claim 5, wherein a content of the water-soluble functional component with respect to a total amount of the external agent for skin is 0.001% by mass or more and 50% by mass or less.

7. The external agent for skin or mucous membrane according to claim 5, wherein the water-soluble functional component is a functional component of cosmetics and/or a functional component of pharmaceuticals.

8. The external agent for skin or mucous membrane according to claim 1, wherein a content of the oil phase with respect to a total amount of the external agent for skin is 70% by mass or less.

9. The external agent for skin or mucous membrane according to claim 1, wherein a content of the petrolatum with respect to a total amount of the external agent for skin or mucous membrane is 2% by mass or more and 70% by mass or less.

10.-11. (canceled)

12. A method for producing an external agent for skin or mucous membrane, the method comprising:
adding a water-soluble functional component and/or a water-insoluble functional component to a base for an external agent for skin or mucous membrane, and mixing the water-soluble functional component and/or the water-insoluble functional component with the base,
wherein the base comprises:
an oil phase, as an inner phase, that consists of petrolatum or is a liquid or a semi-solid comprising petrolatum with a viscosity of 5000 mPa·s or more at 25° C., and an aqueous phase as an outer phase, and particles of a polycondensation polymer having a hydroxyl group and/or vesicles formed of an amphiphilic substance,
wherein a part of the particles and/or the vesicles are present at an interface between the oil phase and the aqueous phase.

13. A method for producing an external agent for skin or mucous membrane, the method comprising:
adding an oil phase comprising petrolatum and a water-insoluble functional component dropwisely to an aqueous phase comprising dispersed particles of a polycondensation polymer having a hydroxyl group and/or dispersed vesicles formed of an amphiphilic substance, the oil phase being heated to a melting point of the oil phase or higher to melt, and
mixing with stirring the aqueous phase.

14. A base for an external agent for skin or mucous membrane to produce the external agent for skin or mucous membrane by addition of a water-soluble functional component and/or a water-insoluble functional component, the base comprising:
an oil phase, as an inner phase, that consists of petrolatum or is a liquid or a semi-solid comprising petrolatum with a viscosity of 5000 mPa·s or more at 25° C., and an aqueous phase as an outer phase, and
particles of a polycondensation polymer having a hydroxyl group and/or vesicles formed of an amphiphilic substance,
wherein a part of the particles and/or the vesicles are present at an interface between the oil phase and the aqueous phase.

15. The base for an external agent for skin or mucous membrane according to claim 10, wherein the external agent for skin or mucous membrane is an ointment.

16. The external agent for skin or mucous membrane according to claim 2, wherein a content of the water-insoluble functional component with respect to a total amount of the external agent for skin is 50% by mass or less.

17. The external agent for skin or mucous membrane according to claim 2, wherein the water-insoluble functional component is a functional component of cosmetics and/or a functional component of pharmaceuticals.

18. The external agent for skin or mucous membrane according to claim 2, wherein a content of the oil phase with respect to a total amount of the external agent for skin is 70% by mass or less.

19. The external agent for skin or mucous membrane according to claim 5, wherein a content of the oil phase with respect to a total amount of the external agent for skin is 70% by mass or less.

20. The external agent for skin or mucous membrane according to claim 2, wherein a content of the petrolatum with respect to a total amount of the external agent for skin or mucous membrane is 2% by mass or more and 70% by mass or less. (New) The external agent for skin or mucous membrane according to claim 5, wherein a content of the petrolatum with respect to a total amount of the external agent for skin or mucous membrane is 2% by mass or more and 70% by mass or less.
